SEQUENCE LISTING (22 sequences)

SEQ ID NO: 1 - protein, 6 aa, synthetic construct
  VARIANT >6: note = the entire sequence of amino acids 1-6 may be repeated one or more times
VVGGVV

SEQ ID NO: 2 - protein, 6 aa, synthetic construct
  VARIANT >6: note = the entire sequence of amino acids 1-6 may be repeated one or more times
  SITE 1..2: note = D-valine
  SITE 3..4: note = D-glycine
  SITE 5..6: note = D-valine
VVGGVV

SEQ ID NO: 3 - protein, 6 aa, synthetic construct
  VARIANT >6: note = the entire sequence of amino acids 1-6 may be repeated one or more times
  SITE 4: note = D-glycine
  SITE 5..6: note = D-valine
VVGGVV

SEQ ID NO: 4 - protein, 10 aa, synthetic construct
  VARIANT >10: note = the entire sequence of amino acids 1-10 may be repeated one or more times
  SITE 6: note = D-phenylalanine
  SITE 7: note = D-glutamic acid
  SITE 8: note = D-phenylalanine
  SITE 9..10: note = D-glycine
FKFGGFEFGG

SEQ ID NO: 5 - protein, 6 aa, synthetic construct
VVGGVV

SEQ ID NO: 6 - protein, 6 aa, synthetic construct
  SITE 1..2: note = D-valine
  SITE 3..4: note = D-glycine
  SITE 5..6: note = D-valine
VVGGVV

SEQ ID NO: 7 - protein, 6 aa, synthetic construct
  SITE 4: note = D-glycine
  SITE 5..6: note = D-valine
VVGGVV

SEQ ID NO: 8 - protein, 6 aa, synthetic construct
  SITE 4: note = D-glycine
  SITE 5..6: note = D-valine
  MOD_RES 6: note = amidation
VVGGVV

SEQ ID NO: 9 - protein, 8 aa, synthetic construct
  SITE 5..6: note = D-valine
  SITE 7..8: note = D-glycine
VVGGVVGG

SEQ ID NO: 10 - protein, 10 aa, synthetic construct
  SITE 6: note = D-phenylalanine
  SITE 7: note = D-glutamic acid
  SITE 8: note = D-phenylalanine
  SITE 9..10: note = D-glycine
FKFGGFEFGG

SEQ ID NO: 11 - protein, 8 aa, synthetic construct
VVGGVVGG

SEQ ID NO: 12 - protein, 8 aa, synthetic construct
  SITE 1..2: note = D-valine
  SITE 3..4: note = D-glycine
  SITE 5..6: note = D-valine
  SITE 7..8: note = D-glycine
VVGGVVGG

SEQ ID NO: 13 - protein, 6 aa, synthetic construct
MVGGVV

SEQ ID NO: 14 - protein, 6 aa, synthetic construct
  SITE 1: note = D-methionine
  SITE 2: note = D-valine
  SITE 3..4: note = D-glycine
  SITE 5..6: note = D-valine
MVGGVV

SEQ ID NO: 15 - protein, 6 aa, synthetic construct
XVGGVV

SEQ ID NO: 16 - protein, 6 aa, synthetic construct
  SITE 1: note = D-amino acid
  SITE 2: note = D-valine
  SITE 3..4: note = D-glycine
  SITE 5..6: note = D-valine
XVGGVV

SEQ ID NO: 17 - protein, 6 aa, synthetic construct
  MOD_RES 6: note = amidation
VVGGVV

SEQ ID NO: 18 - protein, 6 aa, synthetic construct
  SITE 1..2: note = D-valine
  SITE 3..4: note = D-glycine
  SITE 5..6: note = D-valine
  MOD_RES 6: note = amidation
VVGGVV

SEQ ID NO: 19 - protein, 6 aa, synthetic construct
KLVFFA

SEQ ID NO: 20 - protein, 6 aa, synthetic construct
  SITE 1: note = D-lysine
  SITE 2: note = D-leucine
  SITE 3: note = D-valine
  SITE 4..5: note = D-phenylalanine
  SITE 6: note = D-alanine
KLVFFA

SEQ ID NO: 21 - protein, 6 aa, synthetic construct
  SITE 5..6: note = D-valine
  MOD_RES 6: note = amidation
VVGGVV

SEQ ID NO: 22 - protein, 6 aa, synthetic construct
  SITE 5..6: note = D-valine
VVGGVV

(19) United States
(12) Patent Application Publication
(10) Pub. No.: US 2026/0285904 A1
(43) Pub. Date: Sep. 24, 2026

(54) RIPPLED BETA-SHEETS FROM MIXED CHIRALITY LINEAR AND CYCLIC PEPTIDES

(71) Applicant: The Regents of the University of California, Oakland, CA (US)

(72) Inventors: Jevgenij A. Raskatov, Santa Cruz, CA (US); Amaruka Hazari, Santa Cruz, CA (US)

(21) Appl. No.: 19/458,169

(22) Filed: Jan. 23, 2026

Related U.S. Application Data
(60) Provisional application No. 63/749,528, filed on Jan. 25, 2025.

(51) Int. Cl. C07K 7/06; C07K 1/00; C07K 7/64

(52) U.S. Cl. CPC C07K 7/06; C07K 1/00; C07K 7/64

(57) ABSTRACT

Provided are rippled β-sheets. Also provided are materials comprising the rippled β-sheets, compositions comprising the materials, and methods of making the rippled β-sheets and materials.

### CROSS-REFERENCE TO RELATED APPLICATIONS

This application claims the benefit of U.S. Provisional Patent Application No. 63/749,528, filed Jan. 25, 2025, which application is incorporated herein by reference in its entirety.

### STATEMENT OF GOVERNMENT SUPPORT

This invention was made with government support under AG074954 awarded by the National Institutes of Health. The government has certain rights in the invention.

### INCORPORATION-BY-REFERENCE OF MATERIAL ELECTRONICALLY SUBMITTED

A Sequence Listing is provided herewith as a Sequence Listing XML, “UCSC-415_SEQLIST” created on Jan. 23, 2026 and having a size of 39,216 bytes. The contents of the Sequence Listing XML are incorporated by reference herein in their entirety.

### INTRODUCTION

The rippled-sheet is a unique structural motif and an emerging area within the field of peptide self-assembly. Unlike the more commonly known pleated β-sheet composed of peptide strands of the same chirality, the rippled β-sheet is composed of mirror-image peptide strands assembled in strictly alternating fashion. Despite being predicted over 70 years ago, by Pauling and Corey, the motif remained largely neglected until recently.^{1,2} Studies, conducted independently in the laboratories of Schneider, Nilsson, and Raskatov form the experimental foundation of the field. Rippled β-sheet formation leads to the formation of hydrogels with enhanced properties³⁻⁶, and promotes Amyloid Beta Chiral Inactivation where mirror-image amyloid drives oligomer-to-fibril conversion attenuating oligomer-associated Aβ neurotoxicity.⁷⁻¹⁰ A detailed review of the history of the field, including its unusually long induction phase, was published jointly by the three investigators in 2021.¹¹

Structural characterization of the rippled β-sheet was limited to biophysical methods until 2022 when the first atomic resolution structures were obtained. There, it was demonstrated that racemic mixtures of tripeptides composed of aromatic amino acids (Phe, Tyr and Trp) formed rippled β-sheets.^{12,13} In subsequent work, the crystallographic foundation was expanded to include sequences of greater length (up to 7-mer) and complexity.¹⁴ It was shown that the rippled β-sheet framework could support a wide range of amino acids including charged (Lys and Glu), aliphatic (Leu, Ile, Val, Ala, Gly and Met) and small polar residues (Ser). In total, the number of ripple-genic residues now stands at twelve, more than half the canonical amino acid alphabet.

### SUMMARY

Provided are rippled β-sheets. Also provided are materials comprising the rippled β-sheets, compositions comprising the materials, and methods of making the rippled β-sheets and materials.

### BRIEF DESCRIPTION OF THE FIGURES

FIG. 1A-1D: X-ray crystal structure of the rippled β-sheet formed by VVGGVV (SEQ ID NO: 5) hexapeptide and its enantiomer vvggvv (SEQ ID NO:6), VVGGVV:vvggvv (SEQ ID NO:5 and SEQ ID NO:6, respectively) in a mixture of trifluoroethanol (TFE) and water. (FIG. 1A) A view down the sheet hydrogen-bonding direction reveals rippling of the sheet. L-peptides are shown in green; D-peptides are shown in purple. (FIG. 1B); A view of the sheet face revealing the antiparallel in-register alignment of strands. All backbone amides are involved in stabilizing the rippled sheet except for the amide linkage between G3 and G4, which is rotated out of the plane of the rippled sheet. (FIG. 1C) Crystal packing viewed along the H-bonding direction reveals gaps between sheet faces. The unit cell is outlined in black. (FIG. 1D) The gaps between sheet faces are filled by water molecules (orange color).

FIG. 2A-2D: X-ray crystal structure of the rippled β-sheet formed by VVGGVV-NH₂ (SEQ ID NO: 17) hexapeptide and its enantiomer vvggvv-NH₂ (SEQ ID NO:18), VVGGVV-NH₂:vVggvv-NH₂ (SEQ ID NO:17 and SEQ ID NO:18, respectively) in a mixture of pentafluoropriopionic acid (PFPA) and water. (FIG. 2A) A view down the sheet hydrogen-bonding direction reveals rippling of the sheet. L-peptides are shown in green; D-peptides are shown in purple. (FIG. 2B) A view of the sheet face revealing the antiparallel out-of-register alignment of strands. All backbone amides are involved in stabilizing the rippled sheet including the amide linkage between G3 and G4. (FIG. 2C) Crystal packing viewed along the H-bonding direction reveals that the rippled sheets adopt a staggered conformation such that V1 and V2 form tight dry interfaces with V5 and V6 of neighboring sheets. The unit cell is outlined. (FIG. 2D) Gaps in packing are partially filled by PFPA molecules (orange color) bound to the N-termini of the peptide strands.

FIG. 3A-3D: X-ray crystal structure of the self-rippling β-sheet formed by VVGgvv-NH₂ (SEQ ID NO:8) hexapeptide in a mixture of hexafluoroisopropanol (HFIP) and water. (FIG. 3A) A view down the sheet hydrogen-bonding direction reveals rippling of the sheet. L-peptides are shown in green; D-peptides are shown in purple. (FIG. 3B) A view of the sheet face revealing the antiparallel in-register alignment of strands. All backbone amides are involved in stabilizing the rippled sheet including the amide linkage between G3 and G4. (FIG. 3C) Crystal packing viewed along the H-bonding direction reveals that the rippled sheets mate at alternating angles of +45 degrees and −45 degrees. The unit cell is outlined. (FIG. 3D) Gaps in packing are filled by HFIP molecules (orange color) bound to the N-termini of the peptide strands.

FIG. 4A-4D: X-ray crystal structure of the pleated β-sheet formed by the cyclic peptide VVGGvvgg (SEQ ID NO:9) in a mixture of hexafluoroisopropanol (HFIP) and water. (FIG. 4A) A view down the sheet hydrogen-bonding direction reveals pleating of the sheet. L-peptides are shown in green; D-peptides are shown in purple. (FIG. 4B) A view of the sheet face revealing in-register parallel stacking of valine residues. Backbone amides V1, G3, g7 and v5 are involved in stabilizing the pleated sheet. (FIG. 4C) Crystal packing viewed along the H-bonding direction shows that the pleated sheets mate face-to-face and are connected by hydrogen-bonding between glycine residues. The unit cell is outlined. (FIG. 4D) Gaps in the packing are filled by water molecules (orange color).

FIG. 5A-5D: X-ray crystal structure of the self-rippling cyclic β-sheet formed by the cyclic peptide FKFGGfefgg (SEQ ID NO:10) in a mixture of dimethylsulfoxide (DMSO) and water. (FIG. 5A) A view down the sheet hydrogen-bonding direction reveals rippling of the sheet. L-peptides are shown in green; D-peptides are shown in purple. (FIG. 5B) A view of the sheet face revealing intra and intermolecular hydrogen bonding. All backbone amides are involved in stabilizing the pleated sheet except for the glycine residues. (FIG. 5C) Crystal packing viewed along the H-bonding direction shows that the rippled sheets are connected by hydrogen-bonding between K3 and E8 residues. The unit cell is outlined. (FIG. 5D) Gaps in the packing are filled by water (yellow color) and DMSO molecules (orange color).

FIG. 6A-6D: The DFT-optimized structures and cohesive energy density of the four systems of cyclic VVGGvvgg (SEQ ID NO:9), (FIG. 6A) pleated parallel β-sheet (FIG. 6B) rippled parallel β-sheet (FIG. 6C) pleated antiparallel β-sheet, and (FIG. 6D) rippled antiparallel β-sheet configuration. The yellow solid lines represent the H-bonds formed on the side of the backbone, while the yellow dashed lines indicate the H-bond that should have formed on the side of the backbone.

FIG. 7A-7D: X-ray crystal structure of the self-sorted pleated β-sheet formed by the cyclic peptide VVGGVVGG (SEQ ID NO:11) and its enantiomer vvggvvgg (SEQ ID NO:12) in a mixture of hexafluoroisopropanol (HFIP) and water. (FIG. 7A) A view down the sheet hydrogen-bonding direction reveals pleating of the sheet. L-peptides are shown in green. (FIG. 7B) A view of the sheet face revealing in-register parallel stacking of valine residues. Backbone amides V1, V6, G3 and G7 are involved in stabilizing the pleated sheet. (FIG. 7C) Crystal packing viewed along the H-bonding direction shows that the pleated sheets mate face-to-face and are connected by hydrogen-bonding between glycine residues. The unit cell is outlined. (FIG. 7D) Gaps in the packing are filled by water molecules (yellow color).

FIG. 8: A comparison of the three-dimensional lattices of VVGGVV:vvggvv (SEQ ID NO:5 and SEQ ID NO:6, respectively) and MVGGVV:mvggvv (SEQ ID NO:13 and SEQ ID NO:14, respectively) HFIP.

FIG. 9: A comparison of the hydrogen bonding between individual strands of the rippled dimers of VVGGVV-NH₂:vvggvv-NH₂ (SEQ ID NO:17 and SEQ ID NO:18, respectively), VVGGVV:vvggvv (SEQ ID NO: 5 and SEQ ID NO: 6, respectively) and MVGGVV:mvggvv (SEQ ID NO: 13 and SEQ ID NO: 14, respectively) HFIP.

FIG. 10: A comparison of the three-dimensional lattices of XVGGVV:xvggvv (SEQ ID NO: 15 and SEQ ID NO: 16, respectively) type structures.

FIG. 11: A comparison of the backbone configurations of XVGGVV:xvggvv (SEQ ID NO: 15 and SEQ ID NO: 16, respectively) type structures and VVGgvv-NH₂ (SEQ ID NO:8).

FIG. 12: Data collocation and refinement statistics.

### DETAILED DESCRIPTION

Before the rippled β-sheets, materials and methods of the present disclosure are described in greater detail, it is to be understood that the rippled β-sheets, materials and methods are not limited to particular embodiments described, as such may, of course, vary. It is also to be understood that the terminology used herein is for the purpose of describing particular embodiments only, and is not intended to be limiting, since the scope of the rippled β-sheets, materials and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the rippled β-sheets, materials and methods. The upper and lower limits of these smaller ranges may independently be included in the smaller ranges and are also encompassed within the rippled β-sheets, materials and methods, subject to any specifically excluded limit in the stated range. Where the stated range includes one or both of the limits, ranges excluding either or both of those included limits are also included in the rippled β-sheets, materials and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the rippled β-sheets, materials and methods belong. Although any rippled β-sheets, materials and methods similar or equivalent to those described herein can also be used in the practice or testing of the rippled β-sheets, materials and methods, representative illustrative rippled β-sheets, materials and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present rippled β-sheets, materials and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the rippled β-sheets, materials and methods, which are, for clarity, described in the context of separate embodiments, may also be provided in combination in a single embodiment. Conversely, various features of the rippled β-sheets, materials and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present rippled β-sheets, materials and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Rippled β-Sheets and Related Materials and Compositions

Aspects of the present disclosure include rippled β-sheets. In some instances, a rippled β-sheet of the present disclosure comprises (L)-(VVGGVV)ₖ (SEQ ID NO:1) dimerized with (D)-(vvggvv)ₖ (SEQ ID NO:2), wherein k is an integer of 1 or greater. In certain embodiments, a rippled β-sheet of the present disclosure is a single-component rippled β-sheet comprising VVGgvvₖ (SEQ ID NO:3), wherein k is an integer of 1 or greater. According to some embodiments, a rippled β-sheet of the present disclosure is a single-component rippled β-sheet comprising cyclic FKFGGfefggₖ (SEQ ID NO:4), wherein k is an integer of 1 or greater.

The term “amino acid” generally refers to any monomer unit that comprises a substituted or unsubstituted amino group, a substituted or unsubstituted carboxy group, and one or more side chains or groups, or analogs of any of these groups. Exemplary side chains include, e.g., thiol, seleno, sulfonyl, alkyl, aryl, acyl, keto, azido, hydroxyl, hydrazine, cyano, halo, hydrazide, alkenyl, alkynl, ether, borate, boronate, phospho, phosphono, phosphine, heterocyclic, enone, imine, aldehyde, ester, thioacid, hydroxylamine, or any combination of these groups. Other representative amino acids include, but are not limited to, amino acids comprising photoactivatable cross-linkers, metal binding amino acids, spin-labeled amino acids, fluorescent amino acids, metal-containing amino acids, amino acids with novel functional groups, amino acids that covalently or noncovalently interact with other molecules, photocaged and/or photoisomerizable amino acids, radioactive amino acids, amino acids comprising biotin or a biotin analog, glycosylated amino acids, other carbohydrate modified amino acids, amino acids comprising polyethylene glycol or polyether, heavy atom substituted amino acids, chemically cleavable and/or photocleavable amino acids, carbon-linked sugar-containing amino acids, redox-active amino acids, amino thioacid containing amino acids, and amino acids comprising one or more toxic moieties.

The term “amino acid” includes, but is not limited to, naturally-occurring α-amino acids and their stereoisomers. “Stereoisomers” of amino acids refer to mirror image isomers of the amino acids, such as L-amino acids or D-amino acids. For example, a stereoisomer of a naturally-occurring amino acid refers to the mirror image isomer of the naturally-occurring amino acid (i.e., the D-amino acid). “L” refers to levorotatory and “D” refers to dextrorotatory.

Naturally-occurring α-amino acids are those encoded by the genetic code as well as those amino acids that are later modified (e.g., hydroxyproline, γ-carboxyglutamate, and O-phosphoserine). Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Amino acids may be referred to herein by either their commonly known three letter symbols or by the one-letter symbols recommended by the IUPAC-IUB Commission on Biochemical Nomenclature. For example, an L-amino acid may be represented herein by its commonly known three letter symbol (e.g., Met for L-methionine) or by an upper-case one-letter amino acid symbol (e.g., M for L-methionine). A D-amino acid may be represented herein by its commonly known three letter symbol (e.g., D-Met for D-methionine) or by a lower-case one-letter amino acid symbol (e.g., m for D-methionine).

The terms “polypeptide,” “peptide,” “protein”, and “polymer of amino acids,” used interchangeably herein, refer to a polymeric form of amino acids of any length (e.g., connected one to the other by peptide bonds between the alpha-amino and carboxy groups of adjacent residues), which can include genetically coded and non-genetically coded amino acids, chemically or biochemically modified or derivatized amino acids, and polypeptides having modified peptide backbones.

According to some embodiments, k is an integer of from 1 to 1000, such as from 1 to 750, from 1 to 500, from 1 to 250, from 1 to 100, from 1 to 75, from 1 to 50, from 1 to 40, from 1 to 30, from 1 to 20, or from 1 to 10.

In certain embodiments, the rippled β-sheets are water-soluble, i.e., capable of being dissolved in water. According to some embodiments, the termini of the monomers of the dimers comprise a moiety that renders the dimers water-soluble. Examples of such moieties include, but are not limited to, a free amine, a free carboxylate, and/or the like.

Aspects of the present disclosure further include materials comprising a plurality of the rippled β-sheets of the present disclosure.

Also provided are compositions comprising the materials of the present disclosure. In certain embodiments, such a composition includes a material of the present disclosure present in a liquid medium, e.g., an aqueous liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino) ethanesulfonic acid sodium salt (MES), 3-(N-Morpholino) propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a protease inhibitor, glycerol, a chelating agent, and the like may be present in such compositions.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the materials against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In certain embodiments, a composition of the present disclosure comprises the material and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the composition, e.g., at concentrations ranging from about 0.001 to about 2% weight/volume (w/v).

### Methods

Also provided by the present disclosure are methods of making and using the rippled β-sheets, materials and compositions of the present disclosure.

According to some embodiments, provided are methods comprising producing a polypeptide comprising, consisting essentially of, or consisting of (L)-(VVGGVV)ₖ (SEQ ID NO:1); and producing a polypeptide comprising, consisting essentially of, or consisting of, (D)-(vvggvv)ₖ (SEQ ID NO:2), wherein k is an integer of 1 or greater.

According to some embodiments, provided are methods comprising producing a polypeptide comprising, consisting essentially of, or consisting of VVGgvvₖ (SEQ ID NO:3), wherein k is an integer of 1 or greater.

According to some embodiments, provided are methods comprising producing a cyclic polypeptide comprising, consisting essentially of, or consisting of FKFGGfefggₖ (SEQ ID NO:4), wherein k is an integer of 1 or greater.

In some instances, the polypeptides are produced by chemical synthesis. A non-limiting example of a chemical synthesis includes solid-phase synthesis (e.g., solid-phase peptide synthesis). Solid-phase peptide synthesis (SPPS) involves the successive addition of protected amino acid derivatives to a growing peptide chain immobilized on a solid phase, including deprotection and washing steps to remove unreacted groups and also side products. Any number of solid supports may be employed, including resins such as polystyrene and polyamide based resins. The peptides may be covalently bound to a solid support, typically at their C-terminal end through linkers such as acid labile and photolabile linkers. In some embodiments the linker is an acid labile linker. In other embodiments, the linker is a trityl linker such as a 2-chlorotrityl linker. Peptide synthesis is typically performed by coupling a protected amino acid to the N-terminal end of the bound sample. The protected amino acid may contain N-terminal protecting groups such as a Boc (tert-butyloxycarbonyl) or Fmoc (9-fluorenylmethyloxycarbonyl) group as well as side chain protecting groups. According to some embodiments, when solid-phase polypeptide synthesis is performed to produce the polypeptides, the solid-phase synthesis is Fmoc-based solid-phase synthesis.

The above-described methods may further comprise purifying the produced polypeptides. Any suitable approach for purifying the polypeptides may be employed. In certain embodiments, the polypeptides are purified by chromatography, a non-limiting example of which is High Performance Liquid Chromatography (HPLC). An example HPLC-based approach suitable for purifying the polypeptides is described in Warner et al. (2017) JoVE, 2017, e55482.

The above-described methods may further comprise combining the produced polypeptides into a racemic mixture. In certain embodiments, the combining is under conditions suitable for formation of the rippled β-sheets of the present disclosure. For example, according to some embodiments, provided are methods that comprise combining the L- and D-polypeptides (e.g., (L)-(VVGGVV)ₖ (SEQ ID NO:1) and (D)-(vvggvv)ₖ (SEQ ID NO:2), wherein k is an integer of 1 or greater) in a mixture under conditions in which the rippled β-sheets are formed. Non-limiting examples of suitable conditions for forming the rippled β-sheets of the present disclosure are described in detail herein, e.g., in the Experimental section below.

According to some embodiments, the conditions comprise combining the polypeptides in the presence of a fluorinated solvent. Non-limiting examples of fluorinated solvents which may be employed when practicing the methods of the present disclosure include hexafluoroisopropanol (HFIP) and pentafluoropropionic acid (PFPA).

Aspects of the present disclosure include combining VVGgvvₖ (SEQ ID NO:3) under conditions in which rippled β-sheets are formed, wherein k is an integer of 1 or greater. Further aspects of the present disclosure include combining cyclic FKFGGfefggₖ (SEQ ID NO:4) under conditions in which rippled β-sheets are formed, wherein k is an integer of 1 or greater.

Non-limiting aspects and embodiments of the present disclosure are further disclosed in the following numbered clauses.

1. A rippled β-sheet comprising (L)-(VVGGVV)ₖ (SEQ ID NO:1) dimerized with (D)-(vvggvv)ₖ (SEQ ID NO:2), wherein k is an integer of 1 or greater.

2. The rippled β-sheet of clause 1, wherein the C-termini of (L)-(VVGGVV)ₖ (SEQ ID NO:1) and (D)-(vvggvv)ₖ (SEQ ID NO:2) are amidated.

3. A single-component rippled β-sheet comprising VVGgvvₖ (SEQ ID NO:3), wherein k is an integer of 1 or greater.

4. The rippled β-sheet of clause 3, wherein the C-termini of VVGgvvₖ (SEQ ID NO:3) are amidated.

5. A single-component rippled β-sheet comprising cyclic FKFGGfefggₖ (SEQ ID NO:4), wherein k is an integer of 1 or greater.

6. A material comprising a plurality of the rippled β-sheets of any one of clauses 1-5

7. A composition comprising the material of clause 6.

8. The composition of clause 7, wherein the material is present in a liquid medium.

9. A method comprising:
producing a polypeptide comprising, consisting essentially of, or consisting of (L)-(VVGGVV)ₖ (SEQ ID NO:1); and
producing a polypeptide comprising, consisting essentially of, or consisting of, (D)-(vvggvv)ₖ (SEQ ID NO:2), wherein k is an integer of 1 or greater.

10. The method of clause 9, further comprising combining the polypeptides in a mixture under conditions in which rippled β-sheets are formed.

11. A method comprising combining VVGgvvₖ (SEQ ID NO:3) under conditions in which rippled β-sheets are formed, wherein k is an integer of 1 or greater.

12. A method comprising combining cyclic FKFGGfefggₖ (SEQ ID NO:4) under conditions in which rippled β-sheets are formed, wherein k is an integer of 1 or greater.

The following examples are offered by way of illustration and not by way of limitation.

### EXPERIMENTAL

### Example 1—Formation of Rippled β-Sheets from Mixed Chirality Linear and Cyclic Peptides

Previous work demonstrated that the rippled β-sheets formed from MVGGVV (SEQ ID NO: 13) and its enantiomer mvggvv (SEQ ID NO:14) included solvent in the interface between sheets.¹⁴ The inclusion of solvent in the interface led to fibril architectures distinct from the dry-interfaces typically observed in enantiopure amyloid-like crystals. A comparison of the crystal structures of enantiopure MVGGVV¹⁶ (SEQ ID NO:13) and racemic MVGGVV:mvggvv (SEQ ID NO: 13 and SEQ ID NO:14, respectively) revealed that the central diglycine bridge is highly flexible. Accordingly, this feature was incorporated into the present designs of linear, cyclic and single-component mixed chirality peptides.

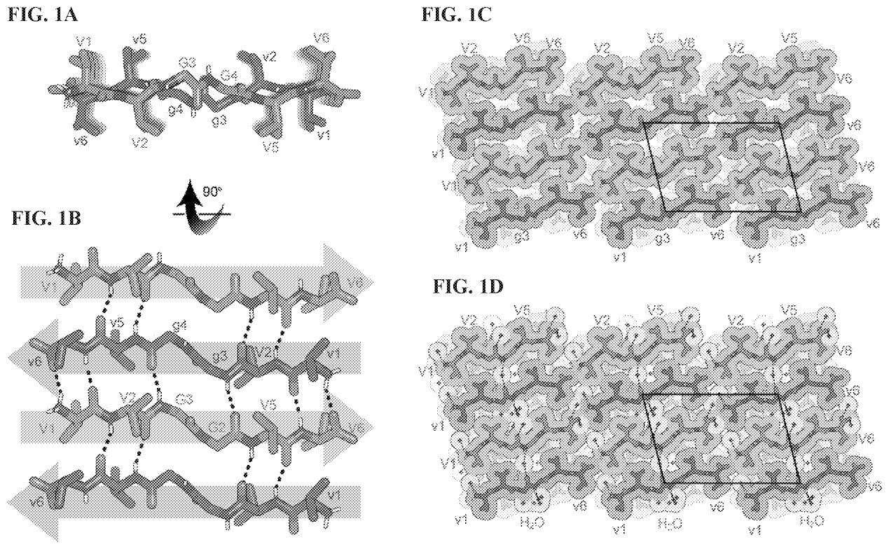

To examine the effect of a single amino acid modification on rippled-sheet structure, the methionine residue of MVGGVV (SEQ ID NO:13) was replaced with a valine (VVGGVV). A racemic mixture of VVGGVV (SEQ ID NO:5) and vvggvv (SEQ ID NO:6) was crystallized from a solution of trifluoroethanol (TFE) in water, yielding needles of antiparallel rippled β-sheet layers as determined by X-ray crystallography (FIG. 1A-1B). As observed in the previously reported structures of the MVGGVV:mvggvv (SEQ ID NO:13 and SEQ ID NO:14, respectively) polymorphs¹⁴, the individual hexapeptides stack in the H-bonding dimension, forming extended antiparallel rippled β-sheet layers, in which mirror image peptide strands are arranged in strictly alternating fashion. Each L-hexapeptide (FIG. 1B, green) is sandwiched between two d-hexapeptides (FIG. 1B, purple), and each d-hexapeptide is sandwiched between two L-hexapeptides in periodic fashion, with H-bond distances ranging from 1.99 to 2.97 Å. Each hexapeptide has six H-bonds to one of its two direct neighbors in the layer, forming a tight dimer, and four H-bonds to the other, forming a loose dimer (FIG. 1B). The rippled interface is partially interrupted at the G3-G4 junction. As a consequence, the amide backbone at G3-G4 does not participate in the stabilization of the rippled β-sheet.

The torsional angles for the glycine residues G3 and G4 are measured as φ=−75.4 and ψ=156.6 and, φ=−81.4 and ψ=169.9, respectively for the L-hexapeptide, and φ=75.4 and ψ=−156.6 and, φ=81.4 and ψ=−169.9, respectively, for the inversion-related D-hexapeptide. The G3 and G4 φ and ψ angles are comparable to those found in the structures of both MVGGVV:mvggvv polymorphs¹⁴. As observed in the MVGGVV:mvggvv (SEQ ID NO:13 and SEQ ID NO: 14, respectively) structures, the G3-G4 peptide plane is rotated by ~90 degrees from its standard position in a β-sheet. This rotation positions the V2 and V5 sidechains on the same face of the β-sheet (FIG. 1A).

In the three-dimensional lattice, the rippled β-sheets are packed closely together (5.5 Å) forming a small cavity, which is occupied by a cluster of water molecules (FIG. 1C, FIG. 1D). The water molecules hydrogen bond to the amide backbone of G3 and G4. The valine residues are more tightly packed, forming a dry interface between pairs of sheets.

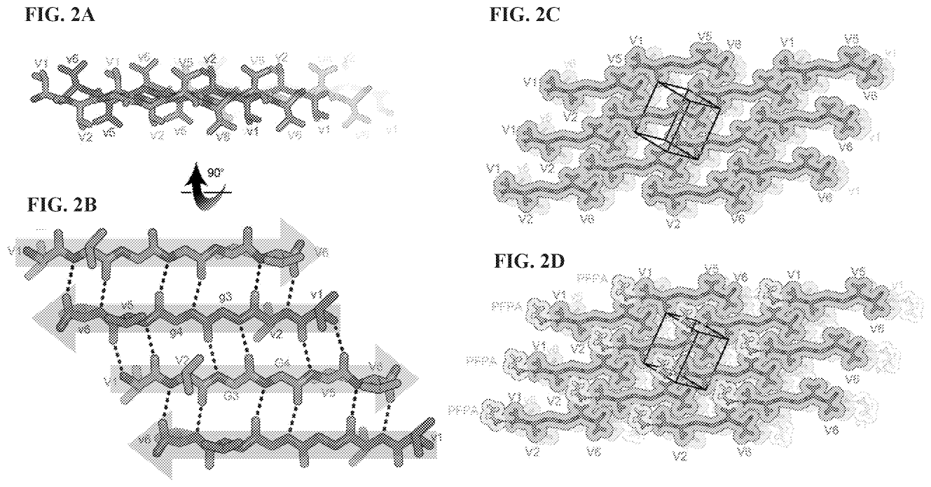

To determine the effect of charge on rippled β-sheet structure, the C-termini of VVGGVV (SEQ ID NO:5) and vvggvv (SEQ ID NO:5) were amidated to form cationic peptides. A racemic mixture of VVGGVV-NH₂ (SEQ ID NO:17) and vvggvv-NH₂ (SEQ ID NO:18) was crystallized from a solution of pentafluoropropionic acid (PFPA) in water, yielding needles of antiparallel rippled β-sheets. The individual hexapeptides stack in the H-bonding dimension, forming extended antiparallel rippled β-sheet layers (FIG. 2A). The hexapeptide strands form an out-of-register sheet resulting in only tight dimers between strands (FIG. 2B). Each hexapeptide strand forms six hydrogen bonds with each of its neighbors with H-bond distances ranging from 1.91 to 2.85 Å.

The torsional angles for the glycine residues G3 and G4 are measured as φ=−150.2 and ψ=160.3, φ=−151.5 and ψ=146.0, respectively for the L-peptide and, φ=150.2 and ψ=−160.3 and, φ=151.5 and ψ=−146.0, respectively for the D-peptide. The flattened configuration has the effect of positioning V2 and V5 sidechains on opposite sides of the β-sheet (FIG. 2A).

The rippled sheets pack face-to-back with a staggered pattern (FIG. 2C) such that V5 and V6 form a tight dry interface with V1 and V2 of a neighboring rippled dimer in the vertical direction. PFPA molecules are bound to the N-termini of the hexapeptides, only partially occupying gaps in the lattice (FIG. 2D).

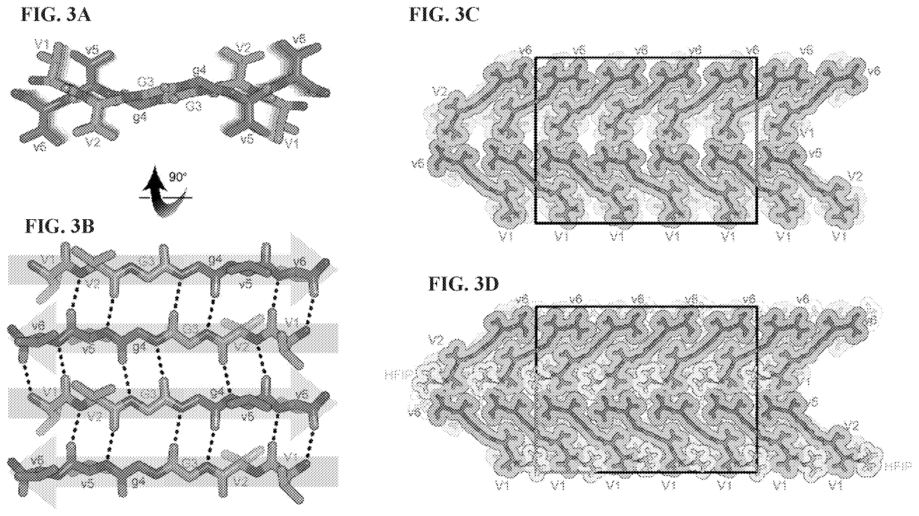

The rippled sheet crystal structures reported here as well as all previously reported structures¹²⁻¹⁴ were constructed from equimolar racemic mixtures of L and D-peptides. Given the success at obtaining atomic resolution structures from XVGGVV:xvggw (SEQ ID NO:15 and SEQ ID NO: 16, respectively) type systems, this framework was used as the basis for the design of a single-component system. The (L,L)-divaline unit was formally connected via diglycine to (D,D)-divaline unit, yielding the peptide with the sequence VVGgvv-NH₂ (SEQ ID NO:8). While the L- and D-segments are not strictly racemic due to their directionality, the system still formed anti-parallel rippled β-sheets upon crystallization from a solution of HFIP in water. Each hexapeptide strand forms 6-H bonds to each of its neighbors with H-bond distances between the rippled dimers ranging from 1.81 to 2.32 Å (FIG. 3A, FIG. 3B). The torsional angles for the glycine residues G3 and G4 are measured as φ=−159.3 and ψ=153.4 and φ=178.9 and ψ=−175.4, respectively. The backbone is highly extended with both G4 φ and ψ angles approaching 180°. When viewed along the hydrogen bonding direction rippled sheets mate at alternating angles of +45 degrees and −45 degrees such that a tight dry interface forms between V1 and V5 and V2 and V6, respectively (FIG. 3C). The gaps in the lattice are occupied by HFIP molecules which bind to the N-terminus of one dimer and the C-terminus of a neighboring dimer, bridging the sheets together (FIG. 3D).

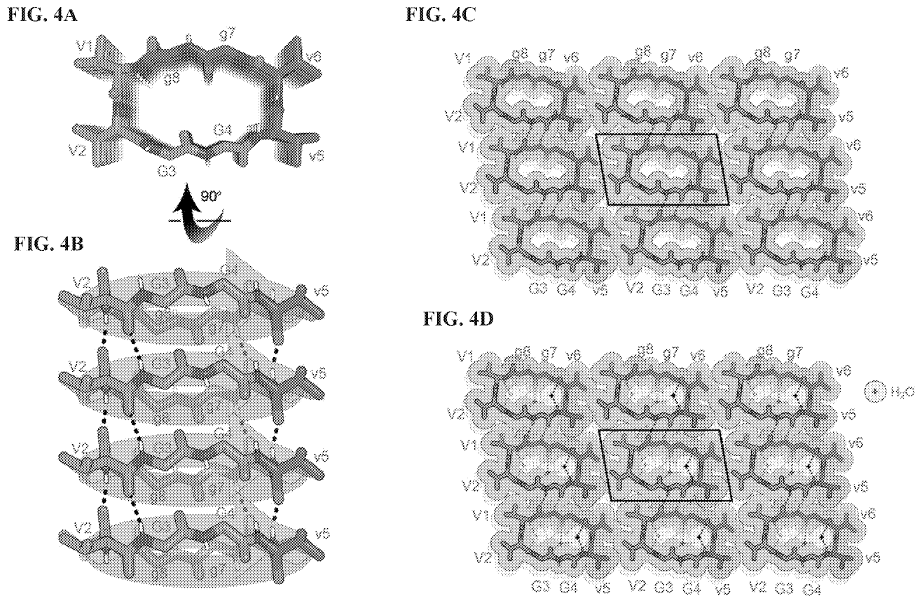

Inspired by this discovery, sought next was to determine whether a cyclic rippled β-sheet could be formed from a single-component system. Designed was a closely related cyclic peptide, the N-to-C cyclized octapeptide, VVGGvvgg (SEQ ID NO:9), which is truly internally racemic (FIG. 4A). Crystals of cyclic VVGGvvgg (SEQ ID NO:9) were grown from a solution of dimethylsulfoxide (DMSO) in water but did not yield rippled β-sheets. Instead, the chiral domains engaged in homochiral self-sorting with L-V1 hydrogen bonding with L-V1, and d-v5 hydrogen bonding with d-v5 (FIG. 4B). In a rippled β-sheet L-Val would be expected to hydrogen bond to D-val. The self-sorted system features a unique hydrogen bonding pattern. The valine residues stack parallel, in-register, whereas the glycine residues form a discontinuous β-sheet which runs at a different angle to the stacked valine residues. Within the β-sheet, 4-H bonds are observed between neighboring cyclic monomers with H-bond distances ranging from 1.90-2.50 Å.

The three-dimensional lattice is stabilized by hydrogen bonding between sheets in the vertical direction; G3 bonds to G8 and G4 bonds to G7, with H bond distances ranging from 2.11-2.20 Å. In the lateral direction, a tight interface between V1 and V6 and V2 and V5 is observed (FIG. 4C). Cavities within the sheets are occupied by water molecules (FIG. 4D).

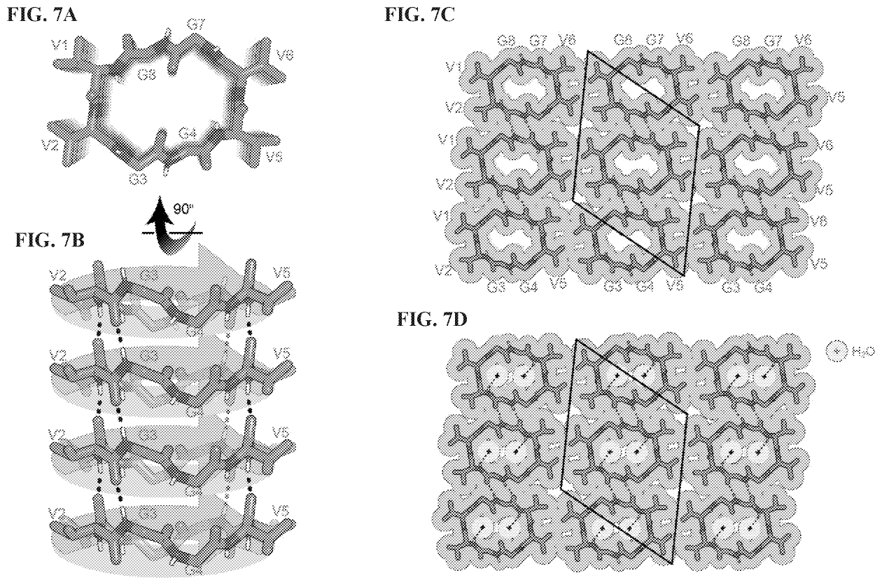

Attempts to form a cyclic rippled sheet from a racemic mixture of cyclic VVGGVVGG (SEQ ID NO: 11) and vvggvvgg (SEQ ID NO:12) led to a similar outcome, with the mixture self-sorting into enantiopure pleated sheets, rather than the desired rippled sheets (FIG. 7A-D).

It was hypothesized that a larger decapeptide may be less strained and thus more likely to form cyclic rippled-sheets. A cyclic decapeptide was devised in which the diglycine linking units were used to formally connect the L-tripeptide FKF and the d-tripeptide fef, producing the zwitterionic cyclic framework FKFGGfefgg (SEQ ID NO:10). The tripeptide segments of the cycle were inspired by the studies with aromatic tripeptides but employed charged residues to enhance solubility.

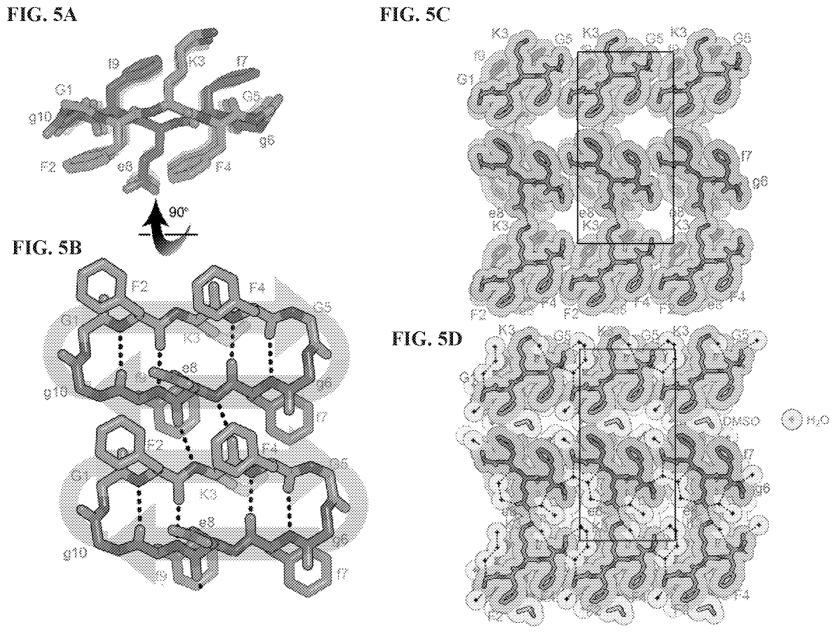

Crystals of the cyclic single-component system FKFGGfefgg (SEQ ID NO:10) were grown from a mixture of DMSO and water and yielded cyclic, self-rippling β-sheets (FIG. 5A). The cyclic peptide forms a rippled monomer which is stabilized by intramolecular hydrogen bonding; 4 hydrogen bonds are observed between the amide backbones of L-F2 and D-f9 and L-F4 and D-f7, ranging from 1.96-2.24 Å. Each rippled monomer forms two hydrogen bonds to a neighboring monomer with H-bonding distances of 2.12 and 2.39 Å (FIG. 5B). The periodic alternation of intra and intermolecular hydrogen bonds results in a tight/loose hydrogen bonding arrangement mirroring that observed in the linear rippled sheet VVGGVV:vvggvv (SEQ ID NO:5 and SEQ ID NO: 6, respectively) (FIG. 1B).

In the three-dimensional lattice the sheets are aligned face-to-face at an angle that enables the oppositely charged termini of residues d-e8 and L-K3 to hydrogen bond and allows a tight interface to form between the phenyl rings of F2 and f9 (FIG. 5C). Clusters of water molecules occupy gaps in the lattice and form hydrogen bonding networks with the termini of D-e8 and L-K3 as well as with oxygen atoms from the amide backbone. Additional gaps in the lattice are filled with DMSO molecules (FIG. 5D).

### Discussion

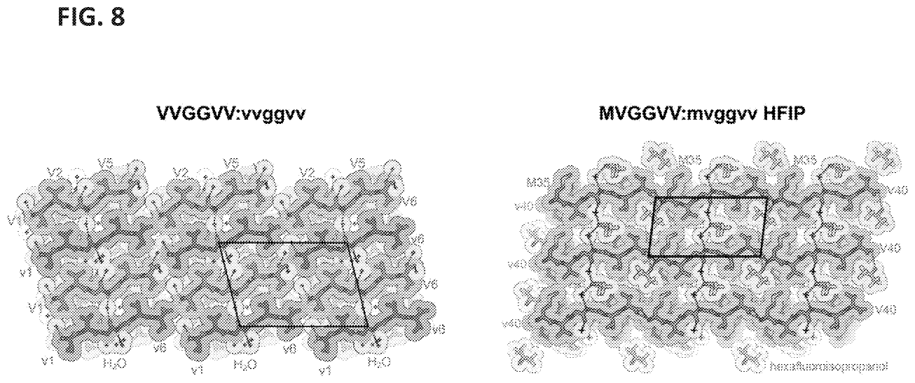

Structural Variation in Racemic and Single-Component Linear Rippled β-Sheets. The results herein show that a single amino-acid modification to the peptide sequence can have a profound impact on the structure of the lattice. While the structures of the rippled dimers of VVGGVV:vvggvv (SEQ ID NO:5 and SEQ ID NO:6, respectively) and the previously reported MVGGVV:mvggvv (SEQ ID NO:13 and SEQ ID NO:14, respectively) polymorphs¹⁴ are alike, the three-dimensional lattices differ. The MVGGVV:mvggvv (SEQ ID NO:13 and SEQ ID NO:14, respectively) structures formed three-dimensional lattices capable of encapsulating fluorinated solvents in the cavities between sheets. In contrast, the rippled β-sheets in VVGGVV:vvggvv (SEQ ID NO:5 and SEQ ID NO:6, respectively) are packed too closely together (5.5 Å vs 10.0 Å in MVGGVV:mvggvv (SEQ ID NO:13 and SEQ ID NO:14, respectively) HFIP) for the cavity between the sheets to accommodate fluorinated solvent (FIG. 8).

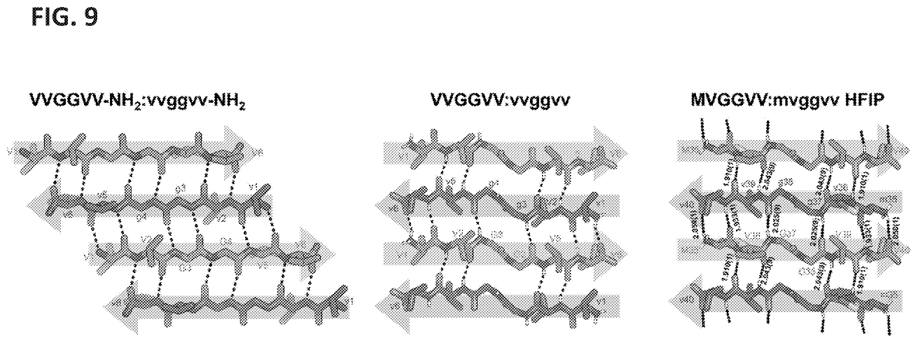

Furthermore, demonstrated herein is that charge can influence the structure of both the rippled dimer and the three-dimensional lattice. A key difference between the cationic system, VVGGVV-NH₂:vvggvv-NH₂, and the zwitterionic systems, VVGGVV:vvggvv (SEQ ID NO:5 and SEQ ID NO:6, respectively) and MVGGVV:mvggvv (SEQ ID NO:13 and SEQ ID NO:14, respectively), is the hydrogen bonding between the individual strands of the rippled dimer; in VVGGVV-NH₂:vvggvv-NH₂ (SEQ ID NO:17 and SEQ ID NO:18, respectively) the hexapeptide strands form an out-of-register sheet resulting in only tight dimers between strands (FIG. 2B) as opposed to the tight/loose dimer arrangement observed in the in-register sheets formed by VVGGVV:vvggvv (SEQ ID NO:5 and SEQ ID NO:6, respectively) (FIG. 1B) and MVGGVV:mvggvv (SEQ ID NO:13 and SEQ ID NO:14, respectively) (FIG. 9). An out-of-register rippled β-sheet was also observed in the crystal structure of the previously reported cationic peptide KLVFFA:klvffa¹⁴ (SEQ ID NO:19 and SEQ ID NO:20, respectively).

An additional distinguishing feature of VVGGVV-NH₂:vvggvv-NH₂ (SEQ ID NO:17 and SEQ ID NO:18, respectively) rippled dimer is the conformation of the backbone. The φ angles in the cationic system vary substantially from those observed in VVGGVV:vvggvv (SEQ ID NO:5 and SEQ ID NO:6, respectively) and MVGGVV:mvggvv (SEQ ID NO:13 and SEQ ID NO:14, respectively), resulting in a more extended backbone conformation at the G3 and G4 residues. Consequently, the V2 and V5 sidechains lie on opposite sides of the β-sheet as opposed to the same side as in VVGGVV:vvggvv (SEQ ID NO:5 and SEQ ID NO:6, respectively) and MVGGVV:mvggvv (SEQ ID NO:13 and SEQ ID NO:14, respectively).

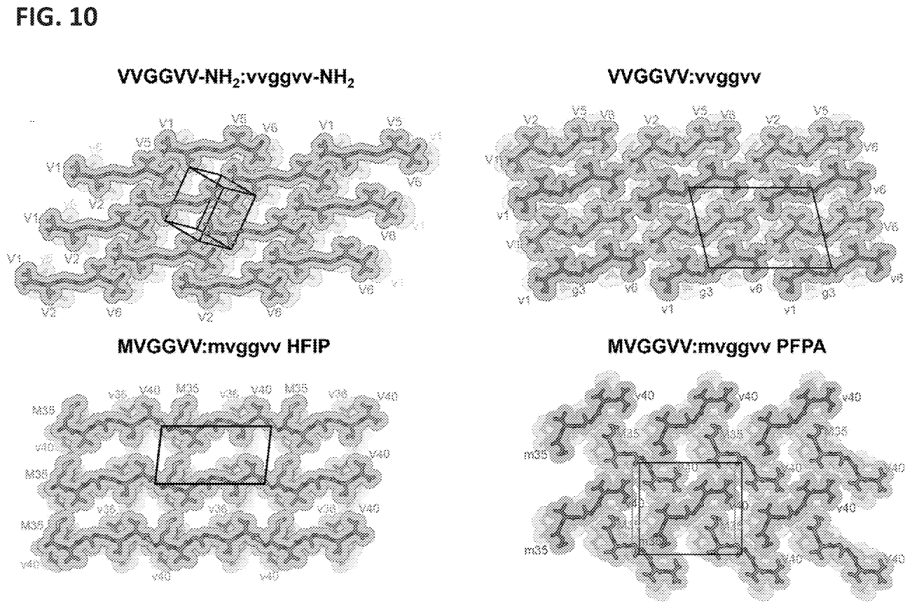

The three-dimensional lattice in VVGGVV-NH₂:vvggvv-NH₂ (SEQ ID NO:17 and SEQ ID NO: 18, respectively) also differs from that observed in VVGGVV:vvggvv (SEQ ID NO:5 and SEQ ID NO: 6, respectively) and MVGGVV:mvggvv (SEQ ID NO:13 and SEQ ID NO:14, respectively). The sheets pack in a staggered pattern as opposed to the face-to-face arrangements seen in VVGGVV:vvggvv (SEQ ID NO:5 and SEQ ID NO:6, respectively) and MVGGVV:mvggvv (SEQ ID NO:13 and SEQ ID NO:14, respectively)-HFIP, respectively and the face-to-edge arrangements observed in MVGGVV:mvggvv (SEQ ID NO:13 and SEQ ID NO:14, respectively)-PFPA (FIG. 10). The staggered pattern and binding of PFPA anions to the N-termini of individual peptide strands results in a more open lattice than observed in other XVGGVV:xvggvv (SEQ ID NO:15 and SEQ ID NO: 16, respectively) type structures.

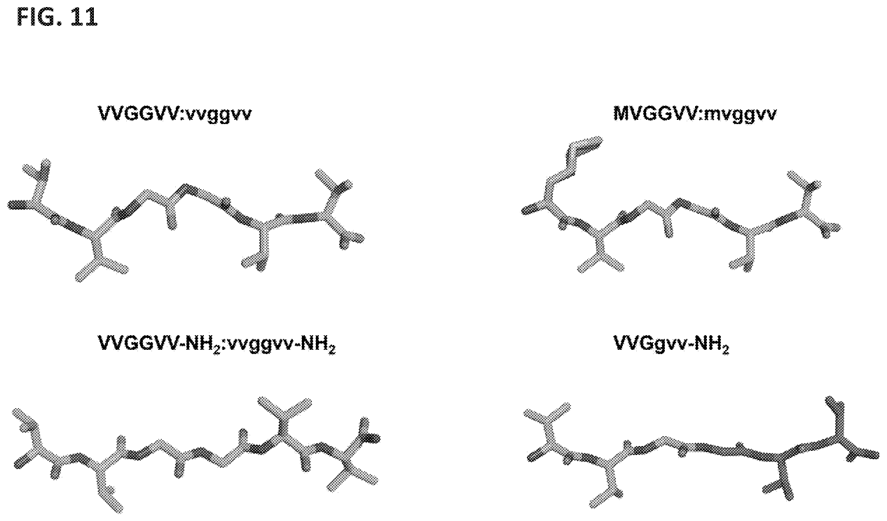

Further structural variation was observed upon analysis of the crystal structure of the single-component system VVGGvv-NH₂ (SEQ ID NO:21). In comparison to the racemic XVGGVV:xvggvv (SEQ ID NO:15 and SEQ ID NO:16, respectively) type structures, the backbone of VVGGvv-NH₂ (SEQ ID NO:21) is more extended with both G4 φ and ψ angles approaching 180° (FIG. 11). The three-dimensional lattice is also distinctive, with rippled sheets mating at alternating angles of +45 degrees and −45 degrees.

It is important to note that while the three linear rippled β-sheet systems described here are composed of the same amino-acid sequence, the two and three-dimensional lattices formed by each system are distinct. In addition to charge and chirality, the diglycine bridge is a contributing element, adopting a range of angles which influence the structure of both the rippled dimer and the three-dimensional lattice.

Computational Analysis of the VVGGvvgg (SEQ ID NO:9) Cyclic Octapeptide. It was interesting to discover that the cyclic octapeptide VVGGvvgg (SEQ ID NO:9) formed pleated β-sheets rather than rippled β-sheets despite the sequence similarity with the “self-rippling” linear system VVGGvv (SEQ ID NO:22). These observations are consistent with a recent solution state NMR study that shows that certain cyclic peptides favor pleated β-sheets over rippled β-sheets.¹⁷ The self-sorting behavior of cyclic VVGGvvgg (SEQ ID NO:9) offered an opportunity to expand the theoretical understanding of the rippled β-sheet motif.¹⁸ Molecular modelling was used to compare the cohesive energy differences of the experimentally observed pleated arrangement with the hypothetical alternatives: anti-parallel pleated, anti-parallel rippled and parallel rippled.

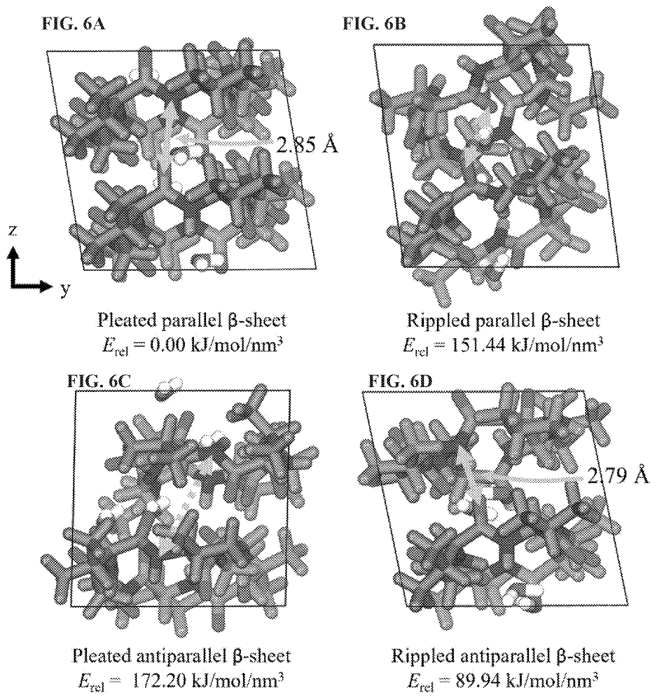

The present results show that the experimentally observed pleated parallel system is the most thermodynamically stable system (FIG. 6A-D). This stability can be attributed to the centrosymmetric arrangement of the cycle, which facilitates H-bond interactions between the sides of the backbone, as indicated by the yellow arrows in FIG. 6A-D. The H-bond interactions between the backbones of the cyclic peptide play a crucial role in stabilizing and maintaining β-sheet structure. Interestingly, the rippled antiparallel system is more thermodynamically stable than the rippled parallel and pleated antiparallel systems. The rippled antiparallel system also features a centrosymmetric arrangement which facilitates hydrogen bonding and allows it to retain its initial structure. The centrosymmetric arrangement is lacking in both the rippled parallel and pleated antiparallel systems, contributing to their lower thermodynamic stability.

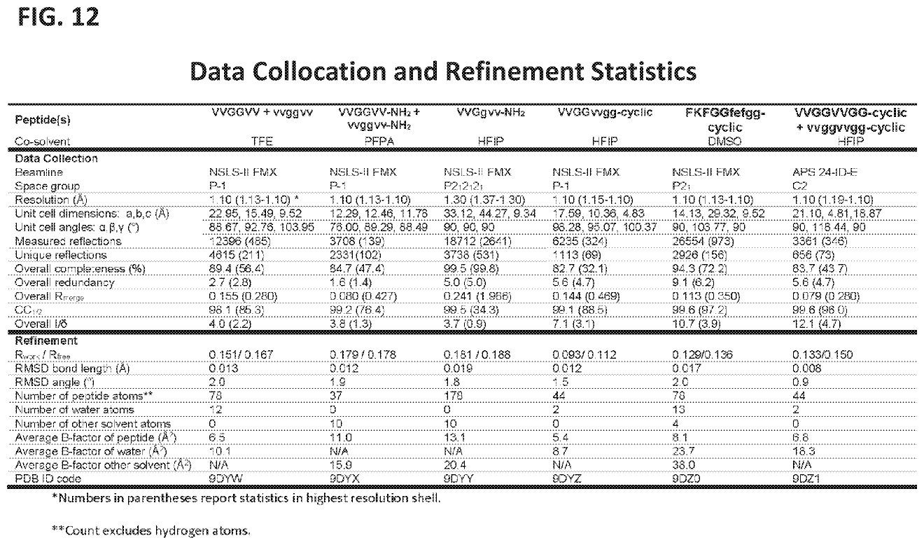

Comparing the lattice parameters from X-ray crystallography with the DFT-optimized cells, the pleated parallel and rippled antiparallel systems show a maximum error of 1.27% and 2.71%, respectively, indicating that the structures are well described (FIG. 12). The rippled parallel and pleated antiparallel systems not observed by crystallography, however, exhibit volume increases of 13.9% and 14.4%, respectively, as the structures shift to increase the number of H-bonds from 0 to 4 between backbones, leading to the disruption of the unit cell.

Ring size vs charge as determinants in cyclic systems. Crystallization of the cyclic octapeptides VVGGvvgg (SEQ ID NO:9) and racemic VVGGVVGG/vvggvvgg (SEQ ID NO:11 and SEQ ID NO:12, respectively) unexpectedly resulted in the formation of pleated β-sheets whereas the cyclic decapeptide FKFGGfefgg (SEQ ID NO:10) formed a rippled β-sheets. Based on these observations, it was hypothesized that ring size is a determinant in whether cyclic peptides prefer pleated or rippled β-sheets; cyclic octapeptides favor pleated sheets whereas decapeptides favor rippled sheets. The role of charge, however, cannot be discounted as cyclic FKFGGfefgg (SEQ ID NO:10) is intrinsically locked into a rippled β-sheet.

### Materials and Methods

### Peptide Synthesis and Crystallization

Peptides were purchased from Anaspec or synthesized on Rink Amide or pre-loaded Wang resins by standard Fmoc based, solid-phase peptide chemistry. Syntheses were performed manually at 0.2 mM scale relative to resin loading. The peptides were cleaved and deprotected with a mixture consisting of trifluoroacetic acid (10 mL), tri-isopropylsilane (1 mL), and liquefied phenol (0.5 mL). The cleavage solution was added to the resins and agitated for 2 h. The solution was then evaporated to 2 mL under nitrogen gas, and the peptides precipitated with cold diethyl ether and centrifuged at 6000 rpm. The peptide pellet was washed with cold diethyl ether, dried, dissolved in 1:1 acetonitrile:water, flash frozen in liquid nitrogen, and lyophilized. No further purification was performed prior to crystallization.

### VVGGVV:Vvggvv (SEQ ID NO:5 and SEQ ID NO:6, Respectively)

Stock solutions of the L-VVGGVV (SEQ ID NO:5) and D-vvggvv (SEQ ID NO:6) peptides in Nanopure water were prepared separately at concentrations of 2 mg/mL. Aliquots (100 μl) of each solution were combined and hexafluoroisopropanol (20 μL) was subsequently added. Colorless needles grew over the course of several weeks.

VVGGVV-NH₂:Vvggvv-NH₂ (SEQ ID NO:17 and SEQ ID NO:18, Respectively)

Stock solutions of the L-VVGGVV-NH₂ (SEQ ID NO:17) and D-vvggvv-NH₂ (SEQ ID NO: 18) peptides in Nanopure water were prepared separately at concentrations of 7 mg/mL. Aliquots (100 ul) of each solution were combined and pentafluoropropionic acid (20 uL) was subsequently added. Colorless needles grew over the course of several weeks.

(SEQ ID NO: 8)
VVGgvv-NH₂

A solution of cyclic VVGGvvgg (SEQ ID NO:9) was prepared by dissolving 1 mg of the peptide in 1 mL of Nanopure water. 100 ul of HFIP was subsequently added. Colorless needles grew over the course of several hours.

Cyclic VVGGVVGG/vvggvvgg

Solutions of cyclic L-VVGGVVGG (SEQ ID NO:11) and D-vvggvvgg (SEQ ID NO:12) were prepared separately by dissolving of 1 mg of each individual peptide in 100 ul of DMSO. The solutions were combined and 1 mL of Nanopure water was then added.

(SEQ ID NO: 9)
Cyclic VVGGvvgg

A solution of cyclic VVGGvvgg (SEQ ID NO:9) was prepared by dissolving 1 mg of the peptide in 100 ul of DMSO. 900 ul of Nanopure water was subsequently added. Colorless needles grew over the course of several hours.

(SEQ ID NO: 10)
Cyclic FKFGGfefgg

A solution of cyclic FKFGGfefgg (SEQ ID NO:10) was prepared by dissolving 1 mg of the peptide in 100 ul of DMSO. 900 ul of Nanopure water was subsequently added. Colorless needles grew over the course of several hours.

### Crystallographic Structure Determination:

Microfocus X-ray beam optics were used to measure crystal diffraction intensities from the crystals since they were needle-shaped, and less than 5 microns thick. Specifically, microfocus beamline 17-ID-2 of the National Synchrotron Light Source-II and beamline 24-ID-E of the Advanced Photon Source located at Argonne National Laboratory were used (Table S1). Crystals were cooled to a temperature of 100 K. Diffraction data were indexed, integrated, scaled, and merged using the programs XDS and XSCALE¹. Data collection statistics are reported in Supplemental Table 1. Phases were obtained by direct methods using the program ShelxD (VVGGVV:vvggvv (SEQ ID NO:5 and SEQ ID NO:6, respectively), VVGGVV-NH₂:vVggvv-NH₂ (SEQ ID NO:17 and SEQ ID NO:18, respectively), VVGgvv-NH₂ (SEQ ID NO:8), and FKFGGfefgg-cyclic (SEQ ID NO:10))² and ShelxT (for VVGGvvgg-cyclic (SEQ ID NO:9) and VVGGVVGG-cyclic:vvggvvgg-cyclic (SEQ ID NO:11 and SEQ ID NO:12, respectively))³. Model building was performed using the graphics program Coot⁴. Atomic refinement was performed using the program Phenix⁵. Subsequent rounds of refinement were performed using the program Refmac⁶ in all cases except VVGGVV-NH₂:vvggvv-NH₂ (SEQ ID NO:17 and SEQ ID NO:18, respectively). Structures were illustrated using PyMOL⁷.

### Computational Details

Density functional theory (DFT) calculations were conducted using Vienna Ab-initio Software Package (VASP) program with the projector-augmented wave (PAW) method. The exchange-correlation interactions were described using the Perdew-Burke-Ernzerhof (PBE) functional, with D3 correction applied to account for dispersion forces. A kinetic energy cut-off 800 eV was used, and the reciprocal space was sampled using (3×3×5) Monkhorst-Pack mesh. The convergence criterion of the self-consistent field iteration was set as 10⁻⁶ eV. Both the cell parameters and atomic positions were fully optimized until the force became less than 10⁻² eV/Å each atom.

### REFERENCES

1. Kabsch W (2010) XDS. Acta Crystallogr D Biol Crystallogr 66:125-132.
2. Sheldrick G M (2008) A short history of SHELX. Acta Crystallogr., A, Found. Crystallogr. 64:112-122.
3. Sheldrick G M. (2015) SHELXT-integrated space-group and crystal-structure determination. Acta Crystallogr A Found Adv. 2015 January; 71 (Pt 1): 3-8. doi: 10.1107/S2053273314026370. Epub 2015 Jan. 1. PMID: 25537383; PMCID: PMC4283466.
4. Emsley P, Lohkamp B, Scott W G, Cowtan K (2010) Features and development of Coot. Acta Crystallogr D Biol Crystallogr 66:486-501.
5. Liebschner D, Afonine P V, Baker M L, Bunkóczi G, Chen V B, Croll T I, Hintze B, Hung L W, Jain S, McCoy A J, Moriarty N W, Oeffner R D, Poon B K, Prisant M G, Read R J, Richardson J S, Richardson D C, Sammito M D, Sobolev O V, Stockwell D H, Terwilliger T C, Urzhumtsev A G, Videau L L, Williams C J, Adams P D. Macromolecular structure determination using X-rays, neutrons and electrons: recent developments in Phenix. Acta Crystallogr D Struct Biol. 2019 Oct. 1; 75(Pt 10):861-877. doi: 10.1107/S2059798319011471. Epub 2019 Oct. 2. PMID: 31588918; PMCID: PMC6778852.
6. Murshudov G N, Skubák P, Lebedev A A, Pannu N S, Steiner R A, Nicholls R A, Winn M D, Long F, Vagin A A (2011) REFMAC 5 for the refinement of macromolecular crystal structures. Acta Crystallogr D Biol Crystallogr 67:355-367.
7. The PyMOL Molecular Graphics System, Version 1.2r3pre, Schrödinger, LLC.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. A rippled β-sheet comprising (L)-(VVGGVV)ₖ (SEQ ID NO:1) dimerized with (D)-(vvggvv)ₖ (SEQ ID NO:2), wherein k is an integer of 1 or greater.

2. The rippled β-sheet of claim 1, wherein the C-termini of (L)-(VVGGVV)ₖ (SEQ ID NO: 1) and (D)-(vvggvv)ₖ (SEQ ID NO:2) are amidated.

3. A single-component rippled β-sheet comprising VVGgvvₖ (SEQ ID NO:3), wherein k is an integer of 1 or greater.

4. The rippled β-sheet of claim 3, wherein the C-termini of VVGgvvₖ (SEQ ID NO:3) are amidated.

5. A single-component rippled β-sheet comprising cyclic FKFGGfefggₖ (SEQ ID NO:4), wherein k is an integer of 1 or greater.

6. A material comprising a plurality of the rippled β-sheets of claim 1.

7. A composition comprising the material of claim 6.

8. The composition of claim 7, wherein the material is present in a liquid medium.

9. A method comprising:
producing a polypeptide comprising, consisting essentially of, or consisting of (L)-(VVGGVV)ₖ (SEQ ID NO:1); and
producing a polypeptide comprising, consisting essentially of, or consisting of, (D)-(vvggvv)ₖ (SEQ ID NO:2),
wherein k is an integer of 1 or greater.

10. The method of claim 9, further comprising combining the polypeptides in a mixture under conditions in which rippled β-sheets are formed.

11. A method comprising combining VVGgvvₖ (SEQ ID NO:3) under conditions in which rippled β-sheets are formed, wherein k is an integer of 1 or greater.

12. A method comprising combining cyclic FKFGGfefggₖ (SEQ ID NO:4) under conditions in which rippled β-sheets are formed, wherein k is an integer of 1 or greater.

13. A material comprising a plurality of the rippled β-sheets of claim 2.

14. A composition comprising the material of claim 13.

15. A material comprising a plurality of the rippled β-sheets of claim 3.

16. A composition comprising the material of claim 15.

17. A material comprising a plurality of the rippled β-sheets of claim 4.

18. A composition comprising the material of claim 17.

19. A material comprising a plurality of the rippled β-sheets of claim 5.

20. A composition comprising the material of claim 19.
